(19) United States
(12) Patent Application Publication
(10) Pub. No.: US 2011/0003898 A1
(43) Pub. Date: Jan. 6, 2011

(54) MEDICAL GLUES FOR SURGERY COMPRISING BIOACTIVE COMPOUNDS

(75) Inventors: Heike Heckroth, Odenthal (DE); Burkhard Köhler, Zierenberg (DE); Sebastian Dörr, Dusseldorf (DE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/920,680

(22) PCT Filed: Feb. 21, 2009
(86) PCT No.: PCT/EP09/01262

§ 371 Date: Sep. 2, 2010

(30) Foreign Application Priority Data
Mar. 6, 2008 (EP) 08004134.6

(51) Int. Cl. A61K 47/00; A61P 17/02; A61P 31/00; A61P 23/02

(52) U.S. Cl. 514/772

(57) ABSTRACT

The present invention relates to novel, rapidly curing adhesives based on hydrophilic polyisocyanate prepolymers for use in surgery, which contain pharmacologically active ingredients. The adhesive system contains
A) isocyanate group-containing prepolymers obtainable from
   A1) aliphatic isocyanates and
   A2) polyols with number-averaged molecular weights of ≧400 g/mol and average OH group contents of from 2 to 6
B) a curing component comprising
   B1) amino group-containing aspartate esters of the general formula (I)

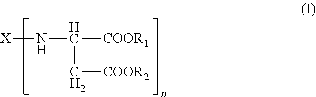

wherein
X is an n-valent organic radical, which is obtained by removal of the primary amino groups of an n-functional amine,
R₁ and R₂ are the same or different organic radicals, which contain no Zerevitinov active hydrogen and
n is a whole number of at least 2 and
   B2) organic fillers which have a viscosity at 23° C. measured to DIN 53019 in the range from 10 to 6000 mPas and
C) optionally reaction products of isocyanate group-containing prepolymers according to the definition of component A) with aspartate esters according to component B1) and/or organic fillers according to component B2) and
D) at least one pharmacologically active compound.

The present invention relates to novel, rapidly curing adhesives based on hydrophilic polyisocyanate prepolymers for use in surgery, which comprise pharmacologically active ingredients.

In recent years, increasing interest has developed in the replacement or complementation of surgical sutures through the use of suitable adhesives. Particularly in the field of plastic surgery, in which particular value is placed on thin, as far as possible invisible scars, adhesives are being increasingly used.

Tissue adhesives must have a number of properties in order to be accepted among surgeons as a substitute for sutures. These include ease of use and an initial viscosity such that the adhesive cannot penetrate into deeper tissue layers or run off. In classical surgery, rapid curing is required, whereas in plastic surgery correction of the adhesive suture should be possible and thus the curing rate should not be too rapid (ca. 5 mins). The adhesive layer should be a flexible, transparent film, which is not degraded in a time period of less than three weeks. The adhesive must be biocompatible and must not display histotoxicity, nor thrombogenicity or potential allergenicity.

Various materials which are used as tissue adhesives are commercially available. These include the cyanoacrylates Dennabond® (octyl 2-cyanoacrylate) and Histoacryl Blue® (butyl cyanoacrylate). However, the rapid curing time and the brittleness of the adhesion site limit their use. Owing to their poor biodegradability, cyanoacrylates are only suitable for external surgical sutures.

As alternatives to the cyanoacrylates, biological adhesives such as peptide-based substances (BioGlue®) or fibrin adhesives (Tissucol) are available. Apart from their high cost, fibrin adhesives are characterized by relatively weak adhesive strength and rapid degradation, so that this is only usable for smaller incisions in untensioned skin.

The isocyanates-containing adhesives described in US 20030135238 and US 20050129733 are based on an aromatic diisocyanate and a hydrophilic polyol, the isocyanates TDI and MDI preferably being used. Both can bear electron-withdrawing substituents in order to increase their reactivity (WO-A 03/9323).

The provision of active substances to the adhesive described therein is of interest for a variety of fields. Using painkillers reduces or eliminates the sensation of pain at the treatment site, thus allowing the subcutaneous injection of a painkiller to be dispensed with. Particularly in the field of veterinary medicine, where topical sections such as castrations or Mulesing in sheep are only rarely carried out using analgesics, a painkiller integrated in the adhesive is indicated. Lowering the sensation of pain also has the effect of reducing the risk of traumatic shock.

The use of substances having antimicrobial/antiseptic activity prevents the penetration of germs into the wound and effects killing of bacteria that are already present. This is especially of interest in veterinary medicine, since only in rare cases is it possible there to work aseptically. The same applies to compounds having antimycotic activity.

The application of bioactive compounds to the intact skin is known in the form of self-adhesive active-substance patches to the skilled worker and is described inter alia in WO 2005/046654, WO 2005/046653 and WO 2004/110428. There, however, the active compound is not integrated in the adhesive. WO 2006/102385 and EP-A 1719530 mention generally the use of bioactive agents in the application of cyanoacrylates and polyurethanes as surgical adhesives.

U.S. Pat. No. 5,684,042, U.S. Pat. No. 5,753,699, U.S. Pat. No. 5,762,919, U.S. Pat. No. 5,783,177, U.S. Pat. No. 5,811,091, U.S. Pat. No. 6,902,594 and EP-A 1508601 describe cyanoacrylates with which, as an antimicrobially active substance, iodine or iodine complexes such as polyvinylpyrrolidone-iodine are used.

US 2003/0007947 describes the use of antimycotics in cyanoacrylate adhesives for the treatment of oral candidiasis; US 2003/0007948 relates to cutaneous candidiasis.

It has now been found that the wound adhesives described in European Patent Applications 07021764.1 and 08001290.9, unpublished at the priority date of the present specification, and based on a combination of hydrophilic aliphatic polyisocyanate prepolymers and aspartates as curing agents, can likewise be provided with active substances, and that the resultant films of adhesive allow release of the active substances.

The subject matter of the present invention is therefore adhesive systems comprising
A) isocyanate group-containing prepolymers obtainable from
   A1) aliphatic isocyanates and
   A2) polyols with number-averaged molecular weights of ≧400 g/mol and average OH group contents of from 2 to 6
B) a curing component comprising
   B1) amino group-containing aspartate esters of the general formula (I)

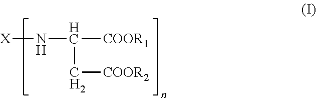

wherein
X is an n-valent organic radical, which is obtained by removal of the primary amino groups of an n-functional amine,
R₁, R₂ are the same or different organic radicals, which contain no Zerevitinov active hydrogen and
n is a whole number of at least 2
   and
B2) organic fillers having a viscosity at 23° C. measured to DIN 53019 in the range from 10 to 6000 mPas
C) where appropriate, reaction products of isocyanate group-containing prepolymers according to the definition of component A) with aspartate esters according to component B1) and/or organic fillers according to component B2)
   and
D) at least one pharmacologically active compound.

For the definition of Zerevitinov active hydrogen, reference is made to Römpp Chemie Lexikon, Georg Thieme Verlag Stuttgart. Preferably, groups with Zerevitinov active hydrogen are understood to mean OH, NH or SH.

In the context of the present invention, tissues are understood to mean associations of cells which consist of cells of the same form and function such as surface tissue (skin), epithelial tissue, myocardial, connective or stromal tissue, muscles, nerves and cartilage. These also include inter alia all organs made up of associations of cells such as the liver, kidneys, lungs, heart, etc.

By pharmacologically active compounds are meant, generally, substances and preparations of substances that are intended for application on or in the human or animal body in order to heal, alleviate, prevent or discern diseases, illnesses, physical damage or complaints. They likewise include substances and preparations for fighting, eliminating or neutralizing pathogens, parasites or exogenous substances.

Analgesics are painkilling substances of various chemical structures and modes of action.

Antiphlogistics are anti-inflammatory substances.

Antiseptics/substances having antimicrobial activity are compounds which inhibit the growth and/or cause the death of certain microorganisms such as, for example, bacteria, fungi, algae and protozoa.

Antimycotics are pharmaceutical agents for the treatment of fungal infections.

Compounds with antiparasitic activity are active substances which kill parasites or inhibit or prevent their growth and also colonization of human or animal tissue.

The isocyanate group-containing prepolymers used in A) are obtainable by reaction of isocyanates with hydroxy group-containing polyols optionally with the addition of catalysts, auxiliary agents and additives.

As isocyanates in A1), for example monomeric aliphatic or cycloaliphatic di- or triisocyanates such as 1,4-butylene diisocyanate (BDI), 1,6-hexamethylene diisocyanate (HDI), isophorone diisocyanate (IPDI), 2,2,4- and/or 2,4,4-trimethylhexamethylene diisocyanate, the isomeric bis-(4,4′-isocyanatocyclohexyl)methanes or mixtures thereof of any isomer content, 1,4-cyclo-hexylene diisocyanate, 4-isocyanatomethyl-1,8-octane diisocyanate (nonane triisocyanate), and alkyl 2,6-diisocyanatohexanoates (lysine diisocyanate) with C1-C8 alkyl groups can be used.

In addition to the aforesaid monomeric isocyanates, higher molecular weight derivatives thereof of uretdione, isocyanurate, urethane, allophanate, biuret, iminooxadiazinedione or oxadiazinetrione structure and mixtures thereof can also be used.

Preferably, isocyanates of the aforesaid nature with exclusively aliphatically or cycloaliphatically bound isocyanate groups or mixtures thereof are used in A1).

The isocyanates or isocyanate mixtures used in A1) preferably have an average NCO group content of from 2 to 4, particularly preferably 2 to 2.6 and quite particularly preferably 2 to 2.4.

In a particularly preferable embodiment, hexamethylene diisocyanate is used in A1).

For synthesis of the prepolymer, essentially all polyhydroxy compounds with 2 or more OH groups per molecule known per se to a person skilled in the art can be used in A2). These can for example be polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols, polyester polycarbonate polyols or any mixtures thereof one with another.

The polyols used in A2) preferably have an average OH group content of from 3 to 4.

Furthermore, the polyols used in A2) preferably have a number-averaged molecular weight of 400 to 20 000 g/mol, particularly preferably 2000 to 10 000 g/mol and quite particularly preferably 4000 to 8500.

Polyether polyols are preferably polyalkylene oxide polyethers based on ethylene oxide and optionally propylene oxide.

These polyether polyols are preferably based on starter molecules with two or more functional groups such as alcohols or amines with two or more functional groups.

Examples of such starters are water (regarded as a diol), ethylene glycol, propylene glycol, butylene glycol, glycerine, TMP, sorbitol, pentaerythritol, triethanolamine, ammonia or ethylenediamine.

Preferred polyalkylene oxide polyethers correspond to those of the aforesaid nature and have a content of ethylene oxide-based units of 50 to 100%, preferably 60 to 90%, based on the overall quantities of alkylene oxide units contained.

Preferred polyester polyols are the polycondensation products, known per se, of di- and optionally tri- and tetraols and di- and optionally tri- and tetracarboxylic acids or hydroxycarboxylic acids or lactones. Instead of the free polycarboxylic acids, the corresponding polycarboxylic acid anhydrides or corresponding polycarboxylate esters of lower alcohols can also be used for the production of the polyesters.

Examples of suitable diols are ethylene glycol, butylene glycol, diethylene glycol, triethylene glycol, polyalkylene glycols such as polyethylene glycol and also 1,2-propanediol, 1,3-propanediol, 1,3-butanediol, 1,4-butanediol, 1,6-hexanediol and isomers, neopentyl glycol or neopentyl glycol hydroxypivalate, with 1,6-hexanediol and isomers, 1,4-butanediol, neopentyl glycol and neopentyl glycol hydroxypivalate being preferred. As well as these, polyols such as trimethylol-propane, glycerine, erythritol, pentaerythritol, trimethylolbenzene or trishydroxyethyl isocyanurate can also be used.

As dicarboxylic acids, phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid can be used. The corresponding anhydrides can also be used as the source of acid.

Provided that the average functional group content of the polyol to be esterified is >2, monocarboxylic acids, such as benzoic acid and hexanecarboxylic acid can also be used as well.

Preferred acids are aliphatic or aromatic acids of the aforesaid nature. Particularly preferred are adipic acid, isophthalic acid and phthalic acid.

Examples of hydroxycarboxylic acids, which can also be used as reaction partners in the production of a polyester polyol with terminal hydroxy groups are hydroxycaproic acid, hydroxybutyric acid, hydroxydecanoic acid, hydroxystearic acid and the like. Suitable lactones are caprolactone, butyrolactone and homologues. Caprolactone is preferred.

Likewise, polycarbonates having hydroxy groups, preferably polycarbonate diols, with number-averaged molecular weights Mₙ of 400 to 8000 g/mol, preferably 600 to 3000 g/mol, can be used. These are obtainable by reaction of carboxylic acid derivatives, such as diphenyl carbonate, dimethyl carbonate or phosgene, with polyols, preferably diols.

Possible examples of such diols are ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethylpentane-1,3-diol, dipropylene glycol, polypropylene glycols, dibutylene glycol, polybutylene glycols, bisphenol A and lactone-modified diols of the aforesaid nature.

Polyether polyols of the aforesaid nature are preferably used for the synthesis of the prepolymer.

For the production of the prepolymer, the compounds of the component A1) are reacted with those of the component A2) preferably with an NCO/OH ratio of 4:1 to 12:1, particularly preferably 8:1, and then the content of unreacted compounds of the component A1) is separated by suitable methods. Thin film distillation is normally used for this, whereby low residual monomer products with residual monomer contents of less than 1 wt. %, preferably less than 0.5 wt. %, quite particularly preferably less than 0.1 wt. %, are obtained.

If necessary, stabilizers such as benzoyl chloride, isophthaloyl chloride, dibutyl phosphate, 3-chloropropionic acid or methyl tosylate can be added during the production process.

The reaction temperature here is 20 to 120° C., preferably 60 to 100° C.

Preferably in formula (I):
R₁ and R₂ are alike or different, optionally branched or cyclic organic radicals having 1 to 20, preferably 1 to 10 carbon atoms, which contain no Zerevitinov active hydrogen,
n is an integer from 2 to 4, and
X is an n-valent organic, optionally branched or cyclic organic, radical having 2 to 20, preferably 5 to 10 carbon atoms, which is obtained by removal of the primary amino groups of an n-valent primary amine.

The production of the amino group-containing polyaspartate ester B1) is effected in a known manner by reaction of the corresponding primary at least bifunctional amine X(NH₂)ₙ with maleate or fumarate esters of the general formula

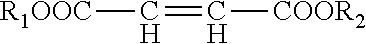

Preferred maleate or fumarate esters are dimethyl maleate, diethyl maleate, dibutyl maleate and the corresponding fumarate esters.

Preferred primary at least bifunctional amines X(NH₂)ₙ are ethylenediamine, 1,2-diaminopropane, 1,4-diaminobutane, 1,3-diaminopentane, 1,5-diaminopentane, 2-methyl-1,5-diaminopentane, 1,6-diaminohexane, 2,5-diamino-2,5-dimethylhexane, 2,2,4- and/or 2,4,4-trimethyl-1,6-diaminohexane, 1,11-diaminoundecane, 1,12-diaminododecane, 1-amino-3,3,5-trimethyl-5-aminomethyl-cyclohexane, 2,4- and/or 2,6-hexahydrotoluoylenediamine, 2,4′- and/or 4,4′-diamino-dicyclohexylmethane, 3,3′-dimethyl-4,4′-diamino-dicyclohexyl-methane, 2,4,4′-triamino-5-methyl-dicyclohexylmethane and polyether amines with aliphatically bound primary amino groups with a number-averaged molecular weight Mₙ of 148 to 6000 g/mol.

Particularly preferred primary at least bifunctional amines are 1,3-diaminopentane, 1,5-diaminopentane, 2-methyl-1,5-diaminopentane, 1,6-diaminohexane and 1,13-diamino-4,7,10-trioxamidecane. Most particular preference is given to 2-methyl-1,5-diaminopentane.

R₁ and R₂ are preferably, independently of one another, C₁ to C₁₀ alkyl radicals, particularly preferably methyl or ethyl radicals.

In a preferred embodiment of the invention, R₁═R₂=ethyl, X being based on 2-methyl-1,5-diaminopentane as the n-functional amine.

Preferably, n in formula (I) for the description of the value of the nth amine is an integer from 2 to 6, particularly preferably 2 to 4.

The production of the amino group-containing aspartate ester B1) from the said starting materials is effected according to DE-A 69 311 633, preferably within the temperature range from 0 to 100° C., the starting materials being used in quantity proportions such that for every primary amino group at least one, preferably exactly one, olefinic double bond is removed, wherein starting materials possibly used in excess can be removed by distillation after the reaction. The reaction can be effected neat or in the presence of suitable solvents such as methanol, ethanol, propanol or dioxan or mixtures of such solvents.

The organic liquid fillers used in B2) are preferably not cytotoxic by cytotoxicity measurements in accordance with ISO 10993.

Examples of organic fillers which can be used are aqueous polyethylene glycols such as PEG 200 to PEG 600, their monoalkyl and dialkyl ethers such as PEG 500 dimethyl ether, aqueous polyether polyols and aqueous polyester polyols, aqueous polyesters such as e.g. Ultramoll (Lanxess AG, Leverkusen, DE) and also glycerol and its aqueous derivatives such as e.g. triacetin (Lanxess AG, Leverkusen, DE).

The organic fillers of component B2) are preferably hydroxy- or amino-functional compounds, preferably purely hydroxy-functional compounds. Preferred purely hydroxy-functional compounds are polyethers and/or polyester polyols, more preferably polyether polyols.

The preferred organic fillers of component B2) possess preferably average OH group contents of 1.5 to 3, more preferably 1.8 to 2.2, very preferably 2.0.

The preferred organic fillers of component B2) preferably possess repeating units derived from ethylene oxide.

The viscosity of the organic fillers of component B2) is preferably 50 to 4000 mPas at 23° C. as measured in accordance with DIN 53019.

In one preferred embodiment of the invention polyethylene glycols are used as organic fillers of component B2). These glycols preferably have a number-average molecular weight of 100 to 1000 g/mol, more preferably 200 to 400 g/mol.

The weight ratio of B1) to B2) is 1:0 to 1:20, preferably 1:0 to 1:12.

The weight ratio of component B2 relative to the total amount of the mixture of B1, B2 and A is situated in the range from 0 to 100%, preferably 0 to 60%.

In order to further reduce the mean equivalent weight of the compounds used overall for prepolymer crosslinking, based on the NCO-reactive groups, in addition to the compounds used in B1) and B2), it is also possible to produce the amino or hydroxyl group-containing reaction products of isocyanate group-containing prepolymers with aspartate esters and/or organic fillers B2), provided that the latter contain amino or hydroxyl groups, in a separate prereaction and then to use these reaction products as a higher molecular weight curing component C).

Preferably, ratios of isocyanate-reactive groups to isocyanate groups of between 50 to 1 and 1.5 to 1, particularly preferably between 15 to 1 and 4 to 1, are used for the pre-extension.

Here, the isocyanate group-containing prepolymer to be used for this can correspond to that of the component A) or else be constituted differently from the components listed as possible components of the isocyanate group-containing prepolymers in the context of this application.

The advantage of this modification by pre-extension is that the equivalent weight and equivalent volume of the curing agent component is modifiable within a clear range. As a result, commercially available 2-chamber dispensing systems can be used for application, in order to obtain an adhesive system which with current chamber volume ratios can be adjusted to the desired ratio of NCO-reactive groups to NCO groups.

Pharmacologically active substances may include, but are not exclusively, the following:
a) analgesics with and without anti-inflammatory activity
b) antiphlogistics
c) substances with antimicrobial activity
d) antimycotics
e) substances having antiparasitic activity

The active substance is preferably soluble at room temperature in the curing agent component B, but may also be used in suspension in B. In a preferable embodiment of the invention the active substance is dissolved or suspended in a mixture of curing agent B1 and filler B2, use being made as B2 preferably of polyethylene glycols having a number-average molecular weight of 100 to 1000 g/mol, more preferably of 200 to 400 g/mol.

The concentration of the active substance added is guided by the therapeutically necessary doses and is 0.001% to 10% by weight, preferably 0.01% to 5% by weight, based on the total amount of all the non-volatile components of the adhesive system.

A feature of all of the active substances that can be employed are that they do not possess NCO-reactive functional groups, or that the reaction of any functional groups present with the isocyanate prepolymer is much slower by comparison with the aspartate/NCO reaction.

Analgesics which fulfil this requirement are local anaesthetics such as ambucaine, amylocalne, arecaidine, benoxinate, benzocaine, betoxycaine, butacaine, butethamine, bupivacaine, butoxycaine, chlorprocaine, cocaethylene, cocaine, cyclomethycaine, dibucaine, dimethocaine, dimethisoquin, etidocaine, fomacaine, isobutyl p-aminobenzoate, leucinocaine, lidocaine, meperidine, mepivacaine, metabutoxycaine, octacaine, orthocaine, oxethazaine, phenacaine, piperocaine, piridocaine, pramoxine, procaine, procain amide, proparacaine, propoxycaine, pseudococaine, pyrrocaine, ropivacaine, tetracaine, tolycaine, tricaine, trimecaine, tropacocaine, amolanone, cinnamoylcocaine, paretoxycaine, propiocaine, myrtecaine and propanocaine.

It is also possible to use opioid analgesics such as morphine and its derivatives (e.g. codeine, diamorphine, dihydrocodeine, hydromorphone, oxycodon, hydrocodon, buprenorphine, nalbuphine, pentazocine), pethidine, levomethadone, tilidine and tramadol.

Equally it is possible to use non-steroidal anti-inflammatory drugs (NSAIDs) such as acetylsalicyl acid, acemetacin, dexketoprofen, diclofenac, aceclophenac, diflunisal, piritramide, etofenamate, felbinac, flurbiprofen, flufeamic acid, ibuprofen, indometacin, ketoprofen, lonazolac, lornoxicam, mefenamic acid, meloxicam, naproxen, piroxicam, tiaprofen acid, tenoxicam, phenylbutazone, propyphenazone, phenazone and etoricoxib. Other analgesics such as azapropazone, metamizole, nabumetone, nefopam, oxacephrol, paracetamol and also the analgesically active amitriptyline can of course likewise be employed.

Besides the stated analgesics which have an anti-inflammatory effect, it is additionally possible to use compounds having a purely anti-inflammatory activity. These include the class of the glucocorticoides such as, for example, cortisone, betamethasone, dexamethasone, hydrocortisone, methylprednisolone, prednisolone, prednisone, budesonide, allotetrahydro-cortisone, fludrocortisone, fluprednisolone, fluticasone propionate, etc.

Substances with an antiseptic activity that can be used include the following compounds among others: triclosan (2,4,4′-trichloro-2′-hydroxydiphenyl ether), chlorhexidine and its salts, octenidine, chloramphenicol, florfenicol, chlorquinaldol, iodine, povidone-iodine, hexachlorophen, merbromine, PHMB, nanocrystalline silver, and also silver salts and copper salts.

As substances with antimicrobial activity it is possible furthermore to use antibiotics from the class of the β-lactams (e.g. penicillin and its derivatives, cephalosporins), tetracyclines (e.g. demeclocycline, doxycycline, oxytetracycline, minocycline, tetracycline), the macrolides (e.g. erythromycin, josamycin, spiramycin), the lincosamides (e.g. clindamycin, lincomycin), the oxazolidinones (e.g. linezolide), the gyrase inhibitors (e.g. danofloxacin, difloxacin, enrofloxacin, ibafloxacin, marbofloxacin, nalidixic acid, pefloxacin, fleroxacin, levofloxacin) and the cyclic peptides (e.g. bicozamycin). It is also possible to use rifamycin, rifaximine, methenamine; mupirocin, fusilic acid, flumequin, and the derivatives of nitroimidazole (e.g. metronidazole, nimorazole, tinidazole), of nitrofuran (furaltadone, nifurpirinol, nihydrazone, nitrofurantoin), of sulfonamide (e.g. sulfabromomethazine, sulfacetamide, sulfachlorpyridazine, sulfadiazine, etc.) and also β-lactamase inhibitors such as clavulanic acid.

As substances with antimycotic activity it is possible to use all azole derivatives which inhibit the biosynthesis of ergosterol, such as, for example, clotrimazole, fluconazole, miconazole, bifonazole, econazole, fenticonazole, isoconazole, oxiconazole, etc. Other antimycotics which can be administered locally are amorolfine, ciclopirox, thymol and its derivatives, and naftifine. The class of the alkylparabens can also be used.

The compounds with antiparasitic activity include inter alia the ectoparasiticides cyfluthrin and lindane, various azole derivatives such as dimetridazole and metronidazole, for example, and also quinine

As and when required, the curing agent component may be stained.

The 2-component adhesive systems according to the invention are obtained by mixing of the prepolymer with the curing components B) and/or C). The biologically active component D) is in components B) and/or C). The ratio of NCO-reactive NH groups to free NCO groups is preferably 1:1.5 to 1:1, particularly preferably 1:1.

Directly after mixing together of the individual components, the 2-component adhesive systems according to the invention preferably have a shear viscosity at 23° C. of 1000 to 10 000 mPas, particularly preferably 2000 to 8000 mPas and quite particularly preferably 2500 to 5000 mPas.

At 23° C., the rate until complete crosslinking and curing of the adhesive is attained is typically 30 secs to 10 mins, preferably 1 min to 8 mins.

A further subject of the invention is the adhesive films obtainable from the adhesive systems according to the invention and laminated parts produced therefrom.

In a preferred embodiment, the 2-component adhesive systems according to the invention are used as tissue adhesives for the closure of wounds in associations of human or animal cells, so that clamping or suturing for closure can to a very large extent be dispensed with.

The tissue adhesives according to the invention can be used both in vivo and also in vitro, with use in vivo, for example for wound treatment after accidents or operations, being preferred.

Hence a process for the closure or binding of cellular tissues, characterized in that the 2-component adhesive systems according to the invention are used, is also an object of the present invention.

Likewise a subject of the invention is the use of such 2-component adhesive systems for the production of an agent for the closure or binding of cellular tissues and the 2-chamber dispensing systems containing the components of the adhesive system fundamental to the invention which are necessary for its application.

### EXAMPLES

Unless otherwise stated, all percentages quoted are based on weight.

As a tissue, beef or pork meat was used for in vitro adhesion. In each case, two pieces of meat (1=4 cm, h=0.3 cm, b=1 cm) were painted at the ends over a 1 cm width with the adhesive and glued overlapping. The stability of the adhesive layer was in each case tested by pulling.

PEG=polyethylene glycol

### Example 1

### Prepolymer A

465 g of HDI and 2.35 g of benzoyl chloride were placed in a 1 l four-necked flask. 931.8 g of a polyether with an ethylene oxide content of 63% and a propylene oxide content of 37% (each based on the total alkylene oxide content) started with TMP (3-functional) were added within 2 hrs at 80° C. and then stirred for a further hour. Next, the excess HDI was distilled off by thin film distillation at 130° C. and 0.1 mm Hg. 980 g (71%) of the prepolymer with an NCO content of 2.53% were obtained. The residual monomer content was <0.03% HDI.

### Example 2

### Aspartate B

1 mol of 2-methyl-1,5-diaminopentane was slowly added dropwise to 2 mols of diethyl maleate under a nitrogen atmosphere, so that the reaction temperature did not exceed 60° C. The mixture was then heated at 60° C. until diethyl maleate was no longer detectable in the reaction mixture. The product was purified by distillation.

### Examples of Tissue Bonding with Active Ingredients

### Example 3

### In Vitro Bonding of Muscular Tissue

0.45 g of PEG 200 were mixed thoroughly with 0.55 g of aspartate B and 2-5% of the active ingredient. The solution was stirred with 4 g of prepolymer A and applied to the tissue. We tested for processing time and the effect of adhesion to meat as well as the formation of a film on the skin.

TABLE 1

| | | Adhesive | Film formation cure time |
|---|---|---|---|
| | Pot | strengths¹ | [min to disappearance of |
| Active substance | life | [after 4 min] | surface tackiness] |
| none | 1 min 10 s | ++ | 3 |
| lidocaine | 1 min 30 s | ++ | 4 |
| acemetacine | 1 min 30 s | + | 7 |
| benzocaine | 1 min 30 s | + | 6 |
| tetracaine | 1 min 40 s | + | 7 |
| phenylbutazone | 1 min 30 s | + | 8 |
| paracetamol | 1 min 30 s | + | 7 |
| ibuprofen | 1 min 30 s | ++ | 6 |
| erythromycin | 1 min 30 s | ++ | 6 |
| nalidixic acid² | 1 min 30 s | ++ | 5 |
| chlorhexidine | 30 s | ++ | 3 |
| triclosan (Irgasan) | 1 min 30 s | ++ | 5 |
| thymol | 1 min 30 s | ++ | 5 |
| fluconazole | 3 min | [3] | 15 |
| metronidazole² | 2 min | ++ | 5 |
| cortisone² | 1 min 20 s | ++ | 5 |
| furaldatone² | 1 min 30 s | + | 4 |
| sulfacetamide | 1 min 15 s | ++ | 5 |
| enrofloxazine² | 1 min | ++ | 4 |
| chloramphenicol | 1 min 30 s | ++ | 5 |
| tetracycline | 1 min 15 s | ++ | 4 |
| acetylsalicylic acid | 1 min 30 s | ++ | 4 |
| amitriptyline | 1 min 20 s | ++ | 3 min 30 s |
| bupivacaine | 1 min 30 s | + | 6 |
| tramadol | 1 min 20 s | ++ | 4 |

¹The adhesive strength was determined by pulling. (++): the pieces of meat could not be separated from one another without fibre tearing, (+): pulling produced tearing in the adhesive layer,
²suspension
[3] adhesive had still not cured

### Example 4

### Active Substance Release

For the quantitative determination of the active substance release, a transparent film with a thickness of 200 [symbol] was produced by knife coating from 4 g of prepolymer A, 0.45 g of PEG 200, 0.55 g of aspartate B and 250 mg of active substance. A section measuring 5×5 cm (weight: 0.5 g) was cut from this film, placed in a petri dish, covered with 20 g of physiological saline solution, and stored in an incubator at 37° C. for 2 h. The quantitative determination was carried out via HPLC-UV/MS (HPLC column: Inertsil ODS 3 5 □ 120 A 125 mm*2.1 mm 60° C.; eluent A: 25 mmol ammonium acetate in water, eluent B: 25 mmol ammonium acetate in methanol). The amount of active substance released quantity is reported in Table 2.

TABLE 2

| | | Amount of active |
|---|---|---|
| Active substance | Release in mg/l | substance released [%] |
| lidocaine | 570 | 45.6 |
| acetylsalicylic acid | 320 | 25.6 |
| phenylbutazone | 355 | 28.4 |
| paracetamol^{[1]} | 136 | 27.2 |
| cortisone^{[1]} | 230 | 46 |
| nalidixic acid^{[1]} | 110 | 22 |
| tetracycline | 547 | 43.8 |
| chloramphenicol | 646 | 51.7 |

^{[1]}100 mg of active substance instead of 250 mg were used in Ex. 4

1.-13. (canceled)

14. An adhesive system comprising
B) isocyanate group-containing prepolymers obtainable from
A1) aliphatic isocyanates and
A2) polyols with number-averaged molecular weights of ≧400 g/mol and average OH group contents of from 2 to 6
B) a curing component comprising
B1) amino group-containing aspartate esters of the general formula (I) 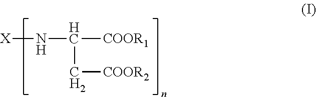
wherein
X is an n-valent organic radical, which is obtained by removal of the primary amino groups of an n-functional amine,
R₁ and R₂ are the same or different organic radicals, which contain no Zerevitinov active hydrogen and
n is a whole number of at least 2
and
B2) organic fillers which have a viscosity at 23° C. measured to DIN 53019 in the range from 10 to 6000 mPas and
C) optionally reaction products of isocyanate group-containing prepolymers according to the definition of component A) with aspartate esters according to component B1) and/or organic fillers according to component B2)
and
D) at least one pharmacologically active compound.

15. The adhesive system according to claim 14, wherein the polyols used in A2) have number-averaged molecular weights of 4,000 to 8,500 g/mol.

16. The adhesive system according to claim 14, wherein a polyalkylene oxide polyether is used in A2).

17. The adhesive system according to claim 14, wherein the organic fillers of component B2) are polyether polyols.

18. The adhesive system according to claim 14, wherein said pharmacologically active compound is an analgesic with or without anti-inflammatory activity, an antiphlogistic, a substance with antimicrobial activity or an antimycotic.

19. An adhesive system comprising
A) isocyanate group-containing prepolymers obtainable from
A1) aliphatic isocyanates and
A2) polyols with number-averaged molecular weights of ≧400 g/mol and average OH group contents of from 2 to 6
B) a curing component comprising
B1) reaction products of isocyanate group-containing prepolymers according to the definition of component A) with an amino group-containing aspartate esters of the general formula (I) 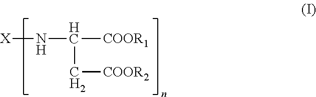
wherein
X is an n-valent organic radical, which is obtained by removal of the primary amino groups of an n-functional amine,
R₁ and R₂ are the same or different organic radicals, which contain no Zerevitinov active hydrogen and
n is a whole number of at least 2 and/or
B2) organic fillers which have a viscosity at 23° C. measured to DIN 53019 in the range from 10 to 6000 mPas
and
C) at least one pharmacologically active compound.

20. The adhesive system according to claim 14, wherein the adhesive system is a tissue adhesive for human or animal tissue.

21. A process for the production of the adhesive system according to claim 14, which comprises mixing components A), B), D) and optionally C) are mixed with one another in a ratio of NCO-reactive groups to free NCO groups of 1:1.5 to 1:1.

22. An adhesive system obtained by the process according to claim 21.

23. A process for the closure or binding of a cellular tissue which comprises utilizing the adhesive systems according to claim 14 to close or bind the cellular tissue.

24. An adhesive film which comprises the adhesive system according to claim 14.

25. A laminated part which comprises the adhesive system according to claim 14.

26. A 2-chamber dispensing system comprising the adhesive system according to claim 14, in which one chamber comprises the prepolymer of component A) and the other comprises the curing component B), active ingredient component D) and optionally component C).
